(19) United States
(12) Patent Application Publication
(10) Pub. No.: US 2026/0174638 A1
(43) Pub. Date: Jun. 25, 2026

(54) TABLETED, SOLID WATERLESS SINGLE USE COSMETIC COMPOSITION

(71) Applicant: ST. JOHN'S UNIVERSITY, Queens, NY (US)

(72) Inventor: Hyunah Cho, Queens, NY (US)

(21) Appl. No.: 18/989,401

(22) Filed: Dec. 20, 2024

(51) Int. Cl. A61K 8/02; A61K 8/60; A61K 8/92; A61K 8/9794; A61Q 19/00; A61Q 19/10

(52) U.S. Cl. CPC A61K 8/0216; A61K 8/60; A61K 8/92; A61K 8/9794; A61Q 19/00; A61Q 19/10; A61K 2800/222

(57) ABSTRACT

The present invention relates to a tableted cosmetic composition containing at least one disintegrating agent, at least one active ingredient and at least one lubricant having a solubility in water of less than 10 mg/L. The tableted cosmetic does not contain water.

### CROSS REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION BY REFERENCE STATEMENT

Not Applicable

STATEMENT REGARDING PRIOR DISCLOSURE BY THE INVENTOR OR A JOINT INVENTOR

Not Applicable

### BACKGROUND OF THE INVENTION

### 1. Field of the Invention

The present invention is related to waterless tablet formulations for single-use cosmetic products. More particularly, the present invention relates to tableted cosmetic composition comprising at least one disintegrating agent and at least one lubricant that is poorly soluble in water, and which does not contain a liquid component.

### 2. Brief Description of the Background Art

WO 2012-104025 relates to a cosmetic containing liquid lipid.

WO 2016-002374 relates to a cosmetic that contains agglomerating agent.

WO 2022-123518 relates to a molded cosmetic mixture that is not pressed.

WO 2022-117858, US 2020/0261327, FR 3117026 and KR 10123093 relate to cosmetics that do not contain any disintegrating agent.

### SUMMARY OF THE INVENTION

Ready-to-use skincare, cosmetic and cleaning products contain more than 85% water. Accordingly, use of reduced amounts of water, or no water, in these products is expected to make a significant beneficial impact on sustainability considering water scarcity expected in the next decades. Additionally, conventional cosmetic products in the forms of liquids and semi-solids require substantial amount of packaging material and storage space. Moreover, ready-to-use cosmetics products contain preservatives to prevent biological contamination, and those supplied in jars and tubs are especially prone to bacterial growth or mold after being opened for use.

Therefore, there is a need for cosmetic products which are water-less, light in weight, appropriate for individual uses and require less storage space and fuel for transportation.

It is an object of the invention to provide a tableted single use cosmetic.

It is another object of the invention to provide a cosmetic that requires less packaging.

It is an additional object of the invention to provide a cosmetic that exhibits improved storage stability.

It is yet another object of the invention to provide a cosmetic that is produced without using water.

It is a further object of the invention to provide a cosmetic that requires less storage space.

These objects and others are achieved by the present invention, which relates to a tableted cosmetic composition comprising at least one disintegrating agent, at least one active ingredient and at least one lubricant having a solubility in water of less than 10 mg/L, and wherein the tableted cosmetic does not contain water.

### BRIEF DESCRIPTION OF THE DRAWINGS

The accompanying drawings, which are incorporated in and constitute a part of the specification, illustrate embodiments of the invention and, together with the description, serve to explain principles of the invention. The patent or application file contains at least one drawing executed in color. Copies of this patent or patent application publication with color drawing(s) will be provided by the Office upon request and payment of the necessary fee.

FIGS. 1A and 1B show examples of water-free tablets according to the present invention.

FIGS. 2A-2F illustrate an embodiment of the present invention as a hand soap.

### DETAILED DESCRIPTION OF THE INVENTION

The present invention relates to a tableted cosmetic composition. The tableted composition comprises at least one disintegrating agent, at least one active cosmetic ingredient, and at least one poorly water-soluble lubricant.

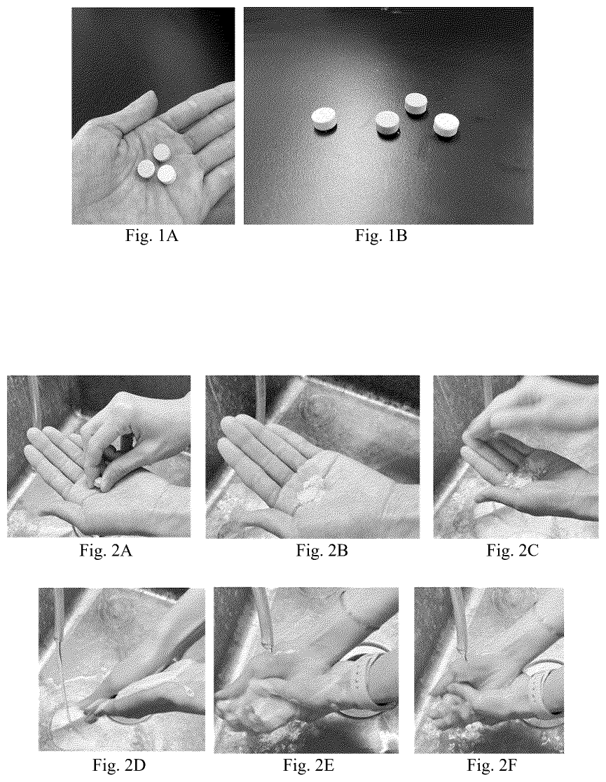

The tableted composition of the present invention is characterized as not containing water or liquid ingredients and is configured to be consumed in a single use. Particularly, the composition comprises one or more disintegrating agents and one or more poorly-water soluble lubricants that ensure rapid disintegration of each tablet when wetted, and which facilitate production using a tablet pressing procedure. Examples of water-free tablets according to the present invention are provided in FIGS. 1A and 1B. In particular embodiments, the form of the composition is a tablet that has a mass of between 200 mg and 2 g, preferably from 300 mg to 2 g.

The tableted composition contains 1-20 total wt % of at least one disintegrating agent. Disintegrating agents remove the binding force caused by high compression and cause the tablet to collapse in water. Most disintegrants have good water absorption and expansion, so as to achieve the disintegration of tablets.

Disintegrants generally operate by enhancing the effect of capillary force and promote water absorption, or by swelling or releasing gas upon contact with water. Disintegration can happen due to the effervescence properties of granules, which can break the tablets very quickly when in contact with water. Swelling of the disintegrant in the presence of water increases the internal pressure of the tablet matrix and cause the disintegration of the tablet.

Disintegrants can increase the porosity and wettability of compressed tablets. Adding disintegrant to tableted compositions increases the surface area of the tableted product and softens the binding substances that hold the solid particles that make up the tablet. In this manner, when the tablet is exposed to water or an aqueous medium, the tablet first decomposes into coarse granules, which then decompose into fine granules. The wetted fine granules dissolve in water, and increase the exposure of the active ingredient to the body, achieving the desired therapeutic effect.

Suitable cosmetically acceptable disintegrating agents are well-known and include, without limitation, starch USP, starch 1500, microcrystalline cellulose, alginic gum, guar gum, croscarmellose sodium, crospovidone, sodium starch glycolate, methylcellulose, sodium carboxymethylcellulose, chitin and chitosan, gum karaya, agar and treated agar, fenugreek seed mucilage, soy polysaccharide, gellan gum, mango peel pectin, lepidium sativum mucilage, plantago seed mucilage, agele marmelos gum, locust bean gum, mangifera indica gum, and hibiscus rosa sinensis mucilage.

Each tablet is designed to dissolve quickly when wetted, and so to readily transform into a lotion by rapid emulsification, or into soap bubbles. Preferably, the tablet is crushed between a user's pointer finger and thumb into a powder in one hand, and combined with a palmful of saline, or tap, purified or distilled water in another hand. The user then rubs both hands together, or mixes the cupped, wetted mixture in their palm with a pointer finger to produce active soap bubbles or to transform the powder into a liquid (e.g., toner, serum) or a semi-solid (e.g., cream).

Preferably, the tableted composition is completely activated using less than 10 ml water. The amount of water utilized to wet each tablet may be 2-10 ml, and the tableted cosmetic is preferably completely activated in less than 5 ml water. An exemplary method, where the tableted composition provides a hand soap, is portrayed in FIGS. 2A-2F. The pH of the completely activated wetted formulation is desirably 5-7.

If reconstitution is not required or desired for administering the active ingredient, tablet compositions may be formulated to self-liquify, e.g., be sufficiently hard for storage but capable of being easily crushable in a palm or between fingertips, with the crushed product being adsorbed directly into the area of contact. This may be preferred, for example, for spot-treating itchy skin.

In further particular embodiments, the inventors contemplate that the tableted cosmetic may be formulated to provide, without limitation, a hand soap, a body soap, a face cleanser, blush, a make-up remover, a face toner, a face serum, a face lotion, a face cream, a face scrub, a face mask, a hair essence or hair serum, a body lotion or a skin irritation relief balm.

Skin care tablets are typically crushed or rubbed between fingers, or between fingers and a palm, and then wetted. Spot treatment tablets may desirably be crushed and rubbed into skin without wetting, as discussed. Soap tablets desirably form detergent bubbles when wetted, and blush products may be personalized by admixing colored tablets with the rehydrated tableted cosmetic, which may then be applied as desired, such as by using a brush.

The tableted composition contains at least one active ingredient. Suitable cosmetically acceptable active agents are well-known and may function as skin moisture enhancers or protectors, and skin improving agents. These skin moisture-enhancing or protecting active agents include, without limitation, carbohydrates, such as mannitol, starch, sucrose, dextrin and maltodextrin. These skin improvement agents include, without limitation, rice, beet, colloidal oat, coconut milk, ginseng, rosemary, green tea, rose petal, mung bean, jojoba oil, olive oil, grapeseed oil, flaxseed oil, mango butter, argan oil, niacinamide, hyaluronic acid and centella asiatica.

The tableted composition contains 2-90 total wt % of at least one active ingredient. Desirable total amounts of active ingredients include 10-90 wt %, 20-90 wt %, 25-90 wt %, 30-90 wt %, 35-90 wt % and 45-90 wt %.

Mannitol may be one preferred active ingredient, since mannitol provides a useful moisturizing effect and also enhances tablet hardness. It is desirable to use 15-30 wt % mannitol since less than 15 wt % mannitol makes the tablet too soft and more than 30 wt % mannitol makes the tablet too hard to crumble readily. More preferred amounts of mannitol are 25-30 wt %.

It is desirable to use 15-35 wt % coconut milk powder since less than 15 wt % coconut milk powder makes the tablet too soft and more than 35 wt % coconut milk powder makes the tablet too milky in appearance and texture when reconstituted. More preferred amounts of coconut milk powder are 20-30 wt %.

It is desirable to use less than 20 wt % colloidal oat since using more than 20 wt % colloidal oat tends to make the tablet too soft and crumbly. It is preferable to use less than 10 wt % colloidal oat and more preferable to use less than 6 wt % colloidal oat.

Surfactants such as sodium cocoyl isothionate are used at less than 14 wt % since greater amounts make the tablet too hard to crumble readily.

The tableted composition contains at least 0.2 total wt % of at least one cosmetically acceptable lubricant having a solubility in water of less than 10 mg/L. Suitable cosmetically acceptable lubricants are well-known and include, without limitation, oil powders, such as cucumber oil powder, bamboo oil powder, dragon fruit oil powder, satsuma orange oil powder, Jasmin oil powder, aloe oil powder, sea kelp oil powder, or white lily oil powder.

It is desirable that the tableted cosmetic contains less than 25 total wt % of lubricant, preferably less than 20 total wt % of lubricant and most preferably less than 15 total wt % of lubricant.

It is understood that powdered oils contained in the tableted cosmetic composition according to the present invention may function as both a lubricant and an active ingredient. For instance, both jojoba oil powder and mango butter powder function as both a lubricant and as an active ingredient. Accordingly, when a particular ingredient is both a lubricant and an active ingredient, that ingredient is included in amounts that satisfy the acceptable ranges for both lubricants and for active ingredients.

Oil powders are prepared by blending oil and powder mixture. The powder mixture is composed of at least one of maltodextrin, guar gum and acacia. The weight ratio of oil to powder mixture is 1 to 20, preferably 2 to 20 and more preferably 5 to 20. The weight ratio of guar gum to the total amount of maltodextrin and acacia is 1 to 1, preferably 5 to 10.

The amount of powdered oil will vary depending upon the intended usage of the tableted cosmetic. Emollients will typically use 4-20 wt % powdered oil since tablets using less than 4 wt % powdered oil function poorly as emollients, and using amounts in excess of 20 wt % make the tablet too soft. Toners will typically include 0.1-10 wt % powdered oil. Creams will include 20-40 wt % powdered oil to achieve a cream-like texture since using less than 20 wt % powdered oil will be too runny when reconstituted, and using more than 40 wt % powdered oil will be too thick and result in insufficient absorbency when reconstituted.

Suitable cosmetically acceptable gelling agents are well-known and may be included in the tableted cosmetic composition of the present invention. A gelling agent is an active ingredient that improves the texture and spreadability of the wet formulation, and enhances skin absorption of the cosmetic. These gelling agents include, without limitation, konjac, acacia and guar gum. Konjac glucomannan is one preferred gelling agent, and is a natural, water-soluble polysaccharide extracted from the tubers of the konjac plant.

Gelling agents are used from 0.1-7 wt % since formulations with more than 7 wt % are too viscous when wetted. 0.1-4 wt % gelling agent is preferred.

Additionally, cosmetically acceptable fragrance or colorants may be included in the tableted cosmetic composition of the present invention. Suitable colorants or fragrances are well-known and include, without limitation, rose petal, green tea, beet root and rosemary.

In particular embodiments, cosmetically acceptable surfactants and effervescent agents may be included in the tableted composition of the present invention. Surfactants are known to be active ingredients which may have anti-wrinkle, anti-redness, cleansing and foaming activities. Suitable cosmetically acceptable surfactants are well-known and include, without limitation, sodium cocoyl iseothionate, and cosmetically acceptable effervescent agents include sodium bicarbonate and citric acid anhydrous.

### Preparation

The solid ingredients, preferably excluding poorly water-soluble ingredients such as lubricant and oil powder are triturated and blended together. Any excluded poorly water-soluble agents are then added to the triturated blended mixture and mixed altogether. The resulting powder mixture is then fed into a tablet press to punch tablets.

Alternatively, each solid ingredient may preferably be triturated separately and pressed into tri-layer tablets, with the outer (first and third) layers comprising active ingredient(s) and the middle layer comprising an excipient layer that prevents physical contact of the two active ingredient layers. Active ingredients include soap, detergent, acid, base, and disintegrant, as well as pre- and pro-biotics, oil, camphor, methanol, kaolin, adsorbent and coloring agent. At least some of soaps, detergents, acids, bases and disintegrants may be found in all formulations, as may be deemed appropriate. Although intended to be without limitation, pre- and pro-biotics and oils may be preferably used in skin care tablets, camphor and menthol may preferably be utilized in spot treatment tablets, and kaolin, adsorbents and coloring agents may preferably be employed in blushes and face powders.

The herein disclosed aspects and embodiments of the invention are further supported by the following non-limiting examples.

### Example 1. A Tablet-Based Hand Soap Formulation (Green Soap)

Detergent: sodium cocoyl isethionate (13 wt %)
Disintegrant (active): Soy polysaccharide (7 wt %)
Effervescence agents (active): Sodium bicarbonate (39 wt %) and citric acid anhydrous (30 wt %)
Lubricating agent: Hydrogenated cotton seed oil powder (1 wt %)
Actives: Rosemary (8 wt %) and green tea (2 wt %)

The solid ingredients were ground and blended until uniform, excluding poorly water-soluble ingredients such as lubricant and oil powder. Hydrogenated cotton seed oil powder was commercially available as a pharmaceutical-grade excipient. The poorly water-soluble agents were added to the mixture later and blended altogether until uniform. The powder mixture was then fed into a tablet press to punch tablets, each weighing 200-2000 mg.

### Example 2. A Tablet-Based Face Toner Formulation (Coconut Milk Toner)

Disintegrant: Soy polysaccharide (4 wt %)
Gelling agent (active): Konjac (4 wt %)
Lubricating agent: Hydrogenated cotton seed oil powder (2 wt %)
Actives: Hyaluronic acid (1 wt %), colloidal oat powder (18 wt %), coconut milk powder (35 wt %), jojoba oil powder (5 wt %) (1 wt % jojoba oil) and mannitol (28 wt %)
Fragrance: Cucumber oil powder 4% (0.8 wt % cucumber oil)

The solid ingredients were ground and blended until uniform, excluding poorly water-soluble ingredients such as lubricant and oil powder. Jojoba oil powder was prepared by blending jojoba oil in maltodextrin in 1:4 w/w ratio. Cucumber oil powder was prepared by blending cucumber oil in maltodextrin in 1:4 w/w ratio. The poorly water-soluble agents were added to the mixture later and blended altogether until uniform. The powder mixture was then fed into a tablet press to punch tablets, each weighing 200-2000 mg.

### Example 3. A Tablet-Based Face Cream Formulation (Mango Butter Cream)

Disintegrant: Soy polysaccharide (4 wt %)
Gelling agent (Active): Konjac (8 wt %)
Lubricating agent: Hydrogenated cotton seed oil powder (2 wt %)
Actives: Jojoba oil powder (16 wt %) (3.2 wt % jojoba oil), Mango butter powder (20 wt %) (2 wt % mango butter), mannitol (27.5 wt %), centella asiatica (1 wt %) and rose petal powder (0.5 wt %)
Fragrance: Dragon fruit oil powder (4 wt %) (0.8 wt % dragon fruit oil)

The solid ingredients were ground and blended until uniform, excluding poorly water-soluble ingredients such as lubricant and oil powder. Jojoba oil powder was prepared by blending jojoba oil in maltodextrin/guar gum mixture in 1:2:2 w/w ratio. Mango butter powder was prepared by blending mango butter in maltodextrin/guar gum mixture in 1:7:3 w/w ratio.

The poorly water-soluble agents were added to the mixture later and blended altogether until uniform. The powder mixture was then fed into a tablet press to punch tablets, each weighing 200-2000 mg.

### Example 4. Hand Soap & Face Wash

Detergent: 0.1-30 wt %
Disintegrating agent: 3-10 wt %
Acid: 20-40 wt %
Base: 25-45 wt %
Lubricating agent: 1-3 wt %
Active: 0.1-25 wt %

The solid ingredients will be ground and blended, excluding poorly water-soluble ingredients such as lubricant and oil powder. Poorly water-soluble agents are added to the mixture later and blended altogether. The powder mixture is then fed into a tablet press to punch tablets, each weighing 200-2000 mg.

### Example 5. Toner

Disintegrant: 3-10 wt %
Gelling agent (active): Konjac 0.1-4 wt %
Lubricating agent: 1-3 wt %
Fragrance: 0.1-10 wt %
Actives: 0.1-96 wt %

The solid ingredients will be ground and blended, excluding poorly water-soluble ingredients such as lubricant and oil powder. Poorly water-soluble agents are added to the mixture later and blended altogether. The powder mixture is then fed into a tablet press to punch tablets, each weighing 200-2000 mg.

### Example 6. Serum

Disintegrant: 3-10 wt %
Gelling agent: Konjac 4-7 wt %
Lubricating agent: 1-3 wt %
Fragrance: 0.1-10 wt %
Actives: 0.1-96 wt %

The solid ingredients will be ground and blended, excluding poorly water-soluble ingredients such as lubricant and oil powder. Poorly water-soluble agents are added to the mixture later and blended altogether. The powder mixture is then fed into a tablet press to punch tablets, each weighing 200-2000 mg.

### Example 7. Cream

Disintegrant: 3-10 wt %
Gelling agent: Konjac 8-10 wt %, guar gum 5 -15 wt %
Lubricating agent: 1-3 wt %
Actives: 0.1-96 wt %

The solid ingredients will be ground and blended, excluding poorly water-soluble ingredients such as lubricant and oil powder. Poorly water-soluble agents are added to the mixture later and blended altogether. The powder mixture is then fed into a tablet press to punch tablets, each weighing 200-2000 mg.

### Example 8. Clay Mask

Kaolin: 50-65 wt %
Actives: 35-50 wt %
Fragrance: 0-3 wt %

The solid ingredients will be ground and blended, excluding poorly water-soluble ingredients such as lubricant and oil powder. Poorly water-soluble agents are added to the mixture later and blended altogether. The powder mixture is then fed into a tablet press to punch tablets, each weighing 200-2000 mg.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

We claim:

1. A tableted cosmetic composition, comprising:
1-20 total wt % of at least one cosmetically acceptable disintegrating agent;
2-90 total wt % of at least one active ingredient; and
at least 0.2 total wt % of at least one cosmetically acceptable lubricant having a solubility in water of less than 10 mg/L, wherein
the tableted cosmetic does not contain water or liquid ingredients.

2. The tableted composition according to claim 1 wherein the tableted cosmetic does not contain any agglomerating agent.

3. The tableted composition according to claim 1, which contains 10-total wt % active ingredient.

4. The tableted composition according to claim 1, which contains 20-total wt % active ingredient.

5. The tableted composition according to claim 1, which contains 25-total wt % active ingredient.

6. The tableted composition according to claim 1, which contains 30-total wt % active ingredient.

7. The tableted composition according to claim 1, which contains 35-total wt % active ingredient.

8. The tableted composition according to claim 1, which contains 45-90 total wt % active ingredient.

9. The tableted composition according to claim 1, which contains less than 30 total wt % of lubricant.

10. The tableted composition according to claim 1, which contains less than 25 total wt % of lubricant.

11. The tableted composition according to claim 1, which contains less than 20 total wt % of lubricant.

12. The tableted composition according to claim 1, which contains less than 15 total wt % of lubricant.

13. The tableted composition according to claim 1, which contains 15-30 wt % mannitol.

14. The tableted composition according to claim 1, which contains 25-30 wt % mannitol.

15. The tableted composition according to claim 1, which contains 20-30 wt % coconut milk powder.

16. The tableted composition according to claim 1, which contains less than 20 wt % colloidal oat.

17. The tableted composition according to claim 1, which contains less than 10 wt % colloidal oat.

18. The tableted composition according to claim 1, which contains less than 6 wt % colloidal oat.

19. The tableted composition according to claim 1, which contains 4-20 wt % powdered oil.

20. The tableted composition according to claim 1, which contains 0.1-10 wt % powdered oil.

21. The tableted composition according to claim 1, which contains 20-40 wt % powdered oil.

22. The tableted composition according to claim 1, which contains 0.1-7 wt % gelling agent.

23. The tableted composition according to claim 1, which contains 0.1-4 wt % gelling agent.

24. A method for preparing the tableted composition according to claim 1, comprising the steps of:
triturating and blending the disintegrating agent and active ingredient;
triturating a powdered oil and blending the triturated powdered oil with the blended triturated disintegrating agent and active ingredient to create a powder mixture; and
feeding the powder mixture into a tablet press to punch tablets, wherein
the tablets weigh 200-2000 mg.

25. A method for preparing the tableted composition according to claim 1, comprising the steps of:
triturating and blending the disintegrating agent and active ingredient to create a first powder;
triturating inactive ingredient to produce a second powder; and
feeding the first and second powders into a tablet press to punch tablets, wherein
the tablets have three layers with two outer layers comprising active ingredient, and an inner layer comprising only inactive ingredients, and
the tablets weigh 200-2000 mg.
